SEQUENCE LISTING (9 sequences)

SEQ ID NO: 1 - DNA, 2615 bp, synthetic construct
  source 1..2615: mol_type = other DNA
ggatccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtcgctcgactcgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagggatctagtggaatgggttctcatcatcatcatcatcatggtatggctagcatgactggtggacagcaaatgggtcgggatctgtacgacgatgacgataaggatctcgccaccatggtcgactcatcccgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtgagtttaaac

SEQ ID NO: 2 - DNA, 9300 bp, synthetic construct
  source 1..9300: mol_type = other DNA
taacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgcgggatccgccaccatggtcttcacactcgaagatttcgttggggactggcgccagacagccggctacaacctagaccaagtccttgaacagggaggtgtgtccagtttgtttcagaatctcggggtgtccgtaactccaatccaaaggattgtcctgagcgatgaaaatgggctgaagatcgacatccatgtcatcatcccatacgaaggtctgagcggcgaccaaatgggccagatcgaaaaaatttttaaggtggtgtaccctgtggatgatcatcactttaaggtgatcctgcactatggcacactggtaatcgacggggttactccaaacatgatcgactatttcggacggccatacgaaggcatcgccgtgttcgacggcaaaaagatcactgtaacagggaccctgtggaacggcaacaaaattatcgacgagcgcctgatcaaccccgacggctccctgctgttccgcgtaaccatcaacggagtgaccggttggagactgtgcgaacgcattctggcgtcgctcgactcgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagggatctagtggaatgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtgagtttaaacggcgcgacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtcccgcgcacatttccccgaaaagtgccacctgac

SEQ ID NO: 3 - DNA, 9361 bp, synthetic construct
  source 1..9361: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagatcgcatgcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatcctctaggtcgacctggatccggtaccgaggagatctgccgccgcgatcgccggcgcgccagatctcaagcttaactagctagcggaccgacgcgtacgcggccgctcgacgattataaggatgacgacgataaattcgtcgagcatcatcaccatcaccattgatgaggtttatccgatccaccggaatgcattctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaatgggcggtagcgtgtacggtgggaggtctatataagcagagctcgtttagtgaaccgtcagaattttgtaatacgactcactatagggcggccgggaattcgccaccatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgcggaagcggagctactaacttcagcctgctgaagcaggctggagacgtggaggagaaccctggacctgtcgacatgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtttaaacgggccggccgcggtctgtacaagtaggattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtcccgcgcacatttccccgaaaagtgccacctgac

SEQ ID NO: 4 - DNA, 39 bp, synthetic construct
  source 1..39: mol_type = other DNA
actagtggaatgggttctcatcatcatcatcatcatggt

SEQ ID NO: 5 - DNA, 31 bp, synthetic construct
  source 1..31: mol_type = other DNA
gtttaaactcacttcgctgtcatcatttgta

SEQ ID NO: 6 - DNA, 32 bp, synthetic construct
  source 1..32: mol_type = other DNA
ggatccatggtcttcacactcgaagatttcgt

SEQ ID NO: 7 - DNA, 35 bp, synthetic construct
  source 1..35: mol_type = other DNA
tctagatcccttgtacagctcgtccatcccaccac

SEQ ID NO: 8 - protein, 4 aa, synthetic construct
SLDS

SEQ ID NO: 9 - protein, 4 aa, synthetic construct
GSSG

(19) United States
(12) Patent Application Publication
(10) Pub. No.: US 2024/0167021 A1
(43) Pub. Date: May 23, 2024

(54) FUSION IMAGING GENE AND LENTIVIRAL EXPRESSION PLASMID, LENTIVIRUS, CELL, PREPARATION METHODS AND APPLICATIONS THEREOF

(71) Applicants: ACADEMY OF MILITARY MEDICAL SCIENCES, ACADEMY OF MILITARY SCIENCES OF CHINESE PLA, Beijing (CN); BEIJING JIUYU ONCOLGY CO., LTD., Beijing (CN)

(72) Inventors: Zhiqiang LIU, Beijing (CN); Zengqiang YUAN, Beijing (CN); Cui WANG, Beijing (CN); Xiaowen XING, Beijing (CN); Bingshui XIU, Beijing (CN); Shihong LIU, Beijing (CN)

(21) Appl. No.: 18/430,589

(22) Filed: Feb. 1, 2024

Related U.S. Application Data
(63) Continuation of application No. PCT/CN2022/080421, filed on Mar. 11, 2022.

(30) Foreign Application Priority Data
Aug. 2, 2021 (CN) 202110882190.4

(51) Int. Cl. C12N 15/10; C12N 15/65; C12N 15/86

(52) U.S. Cl. CPC C12N 15/1086; C12N 15/65; C12N 15/86; C12N 2740/15022; C12N 2740/15043

(57) ABSTRACT

A fusion imaging gene and lentiviral expression plasmid, lentivirus and cell, and preparation methods and applications thereof are provided. The fusion imaging gene includes bioluminescence imaging gene, fluorescent protein gene and calcium imaging gene. The three genes are linked by linkers. The fusion gene is inserted into a modified lentiviral expression plasmid to obtain lentivirus particles carrying the fusion imaging gene.

### CROSS-REFERENCE TO RELATED APPLICATIONS

This application is a continuation of PCT/CN2022/080421, filed on Mar. 11, 2022 and claims priority of Chinese Patent Application No. 202110882190.4, filed on Aug. 2, 2021 the entire contents of which are incorporated herein by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes gas follows:

| | | |
|---|---|---|
| 1. | File name: | Sequence_347-147-PPH_15039 |
| 2. | Creation date: | Jan. 30, 2024 |
| 3. | Byte size: | 30,191 bytes |

### TECHNICAL FIELD

The disclosure relates to the field of biotechnology, and in particular to fusion imaging gene and lentiviral expression plasmid, lentivirus, cell, preparation methods and applications thereof.

### BACKGROUND

Imaging tracing of cell fate has extensive and important applications in the field of biomedical research, such as evaluation of cell response to specific stimuli, evaluation after cell transplantation and so on. Taking cell transplantation as an example, cell fate tracing, including survival, distribution, differentiation and functional integration, is an important index to evaluate safety and effectiveness of the cell. In previous studies, due to the lack of cell lines marked by multi-modal imaging gene, it is impossible to trace the survival/distribution, differentiation/morphology and functional integration of cells after transplantation in the same cell. In order to achieve the above-mentioned different tracing purposes, researchers can only adopt a compromise method, evaluate the survival/distribution, differentiation/morphology and functional integration of seed cells marked by different imaging genes respectively, and comprehensively analyze the fate of cells by synthesizing the tracking data of different marked cells. In a typical report of the study on the integration of transplanted embryonic neurons in the neocortex of the brain in the Nature article in 2016 (Nature, 2016, 539: 248-253), in order to evaluate the structural integration of embryonic neurons in the host, a researchers transplanted neurons marked by red fluorescent protein and detected their structural integration through histopathology. In order to evaluate the functional integration of embryonic neurons in the receptor, neurons marked by Gcamp6f are transplanted, and their functional integration is evaluated by calcium imaging. Finally, the transplantation results of two kinds of cells jointly show that the transplanted embryonic neurons may realize structural and functional integration in the recipient brain. However, the above methods not only increase the cost and workload of research, but also may affect the reliability of the results because the evaluation of structural and functional integration is carried out in independent animals (that is, asynchronous evaluation). For example, functional integration may not occur in structurally integrated cells, and the level of structural integration of cells with functional integration is unknown. Therefore, it is of great significance to synchronously trace different fates of cells.

Obtaining seed cells marked by multi-modal imaging gene is the key to solve the simultaneous evaluation of different fates of cells, which has important application value. Lentivirus is the most commonly used vector for stable gene transfection marking, but there are no reports of lentiviral particles carrying multi-modal imaging gene (quantitative imaging, morphological imaging and functional imaging) and their transfected marked cells. The main reasons are that there are many technical difficulties in achieving the above objectives, including: firstly, the length of multiple reporter genes is large, which is difficult for ordinary lentiviral vectors to accommodate and successfully transfect target cells. In particular, the use of multiple promoters to drive the expression of each reporter gene will further increase the length of foreign genes, making it more difficult to achieve effective packaging of lentivirus particles and successful transfection of target cells; secondly, linking multiple reporter genes to form a fusion multi-modal imaging gene, and using one promoter to drive expression may save the length occupied by the promoter, but improper fusion will lead to functional inactivation due to the spatial interaction between gene products. In addition, the fusion gene may be too long to obtain high-activity lentivirus. It is of great scientific significance and practical value to overcome the problems of inserting large fragments of genes into lentiviruses and maintaining the function of fusion genes, and to construct lentiviruses carrying multi-modal imaging gene for further stable gene marking of multi-modal imaging gene in different cells.

### SUMMARY

The objective of the present disclosure is to provide a lentivirus carrying the fusion multi-modal imaging genes and its application. The lentivirus particles may be transfected to prepare cells stably marked by multi-modal imaging gene, such as human pluripotent stem cells (embryonic stem cell (ESC)/induced pluripotent stem cell (iPSC)), and further, quantitative imaging detection at single cell level, cell morphology detection at histological level and calcium activity function detection at subcellular level may be realized simultaneously for the marked cells themselves and the cells from which they are differentiated.

The above objective of the present disclosure is achieved by the following technical scheme.

The present disclosure relates to fusion imaging gene. The fusion imaging gene includes a bioluminescence imaging gene, a fluorescent protein gene and a calcium imaging gene, and the three genes are linked by linkers.

Preferably, the bioluminescence imaging gene is selected from a Nanoluc gene.

Optionally, the fluorescent protein gene is selected from a mRuby2 gene.

More optionally, the calcium imaging gene is selected from a Gcamp6f gene.

The present disclosure also relates to a lentiviral expression plasmid carrying the fusion imaging gene mentioned above. Optionally, a sequence of the lentiviral expression plasmid carrying fusion imaging gene is shown in SEQ ID NO.2.

The present disclosure also relates to a preparation method of lentiviral expression plasmid, at least including following steps:

S21, modifying the lentiviral expression plasmid,

where the modifying preferably includes:

S211, removing an EF-1α promoter in a pLenti-EF1α-FH-CMV-CopGFP&Puro plasmid by double restriction endonuclease digestion with SphI and BamHI to obtain a skeleton plasmid: a nucleotide sequence of the pLenti-EF1α-FH-CMV-CopGFP&Puro plasmid is shown in SEQ ID NO.3;

S212, inserting a human ubiquitin promoter hUbc into the skeleton plasmid through enzyme link to obtain a pLenti-Ubc-FH-CMV-CopGFP&Puro intermediate plasmid:

S213, removing a sequence from a downstream of Ubc to a Puro gene site in the pLenti-Ubc-FH-CMV-CopGFP&Puro plasmid by PmeI and BamHI double digestion, namely a nucleotide sequence from 3826 to 6112 in the nucleotide sequence shown in the SEQ ID NO.3:

S22, inserting the fusion imaging gene mentioned above into a modified lentiviral expression plasmid to obtain the lentiviral expression plasmid carrying a fusion gene probe.

Optionally, the fusion imaging gene is inserted into the modified intermediate plasmid by using same restriction sites at both ends of the fusion imaging gene to obtain a pLenti-Ubc-Nanoluc-mRuby2-Gcamp6f plasmid, and the lentiviral expression plasmid is obtained through transformation, sequencing, and so on, and a nucleotide sequence of the lentiviral expression plasmid is shown in SEQ ID NO.2.

The present disclosure also relates to a lentivirus carrying the fusion imaging gene mentioned above.

The present disclosure also relates to a preparation method of the lentivirus mentioned above, at least including following steps:
co-transfecting 293T cells by the pLenti-Ubc-Nanoluc-mRuby2-Gcamp6f plasmid and lentivirus packaging plasmids PAX2 and pMD2G to obtain lentivirus particles carrying the fusion imaging gene.

Optionally, a mass ratio of the pLenti-Ubc-Nanoluc-mRuby2-Gcamp6f plasmid, the plasmid PAX2 and the plasmid pMD2G is 3-4: 1.5-2.5: 0.5-1.5, preferably, 3: 2: 1.

The present disclosure also relates to a cell marked by the fusion imaging gene mentioned above or infected by the lentivirus mentioned above. Optionally, the cell is selected from a human H1 embryonic stem cell and a human pluripotent stem cell.

The present disclosure also relates to applications of the cell mentioned above. The applications comprise an application in cell imaging detection: an application in cell fate tracking: or an application in preparing preparations or kits for the cell fate tracking or the cell imaging detection. Optionally, the cell imaging detection is to simultaneously detect cell number imaging, morphological imaging and calcium activity function.

The technical scheme of the disclosure at least includes following beneficial effects.

The lentivirus carrying fusion imaging gene provided by the disclosure may be used for stable multi-modal imaging gene marking of various cells, realizes multi-modal imaging detection of cell number, cell morphology and cell calcium functional activity, simultaneously tracks the different fates of cells, and provides a more powerful tool for cell fate evaluation.

### BRIEF DESCRIPTION OF THE DRAWINGS

FIG. 1 is a schematic structural diagram of fusion multi-modal imaging genes in an embodiment of the present disclosure.

FIG. 2 is an original plasmid map of lentivirus used for modification in an embodiment of the present disclosure.

FIG. 3 is a lentiviral expression plasmid map carrying the fusion multi-modal imaging genes constructed in an embodiment of the present disclosure.

FIG. 4A is a bright field image under phase contrast microscope after the lentiviral expression plasmid carrying the fusion multi-modal imaging genes constructed in an embodiment of the disclosure (the total length of the plasmid <10000 bp) and the packaging plasmid co-transfect 293T cells.

FIG. 4B is a fluorescent microscope image after the lentiviral expression plasmid carrying the fusion multi-modal imaging genes constructed in an embodiment of the disclosure (the total length of the plasmid <10000 bp) and the packaging plasmid co-transfect 293T cells.

FIG. 5A is a bright field image under phase contrast microscope after conventional lentiviral expression plasmid carrying reporter genes (the total length of the plasmid <10000 bp) and the packaging plasmid co-transfect 293T cells in an embodiment of the disclosure.

FIG. 5B is a fluorescent microscope image after conventional lentiviral expression plasmid carrying reporter genes (the total length of the plasmid <10000 bp) and the packaging plasmid co-transfect 293T cells in an embodiment of the disclosure.

FIG. 6A is a bright field image under phase contrast microscope after the conventional lentiviral expression plasmid carrying multiple imaging genes (the total length of the plasmid >11000 bp) and the packaging plasmid co-transfect 293T cells in an embodiment of the disclosure.

FIG. 6B is a fluorescent contrast microscope image after the conventional lentiviral expression plasmid carrying multiple imaging genes (the total length of the plasmid >11000 bp) and the packaging plasmid co-transfect 293T cells in an embodiment of the disclosure.

FIG. 7A is a bright field image under phase contrast microscope of lentivirus particles carrying fusion multi-modal imaging genes transfected into 293T cells in an embodiment of the present disclosure.

FIG. 7B shows an expression of mRuby2 by fluorescent microscope after lentivirus particles carrying the fusion multi-modal imaging genes transfects 293T cells in an embodiment of the present disclosure.

FIG. 7C shows an expression of Gcamp6f by fluorescent microscope after lentivirus particles carrying the fusion multi-modal imaging genes transfects 293T cells in an embodiment of the present disclosure.

FIG. 8A is a bright field image of human embryonic stem cells (hESC) under phase contrast microscope transfected and marked with lentivirus particles carrying fusion multi-modal imaging genes in an embodiment of the disclosure.

FIG. 8B is a fluorescent microscope image of human embryonic stem cells (hESC) transfected and marked with lentivirus particles carrying fusion multi-modal imaging genes in an embodiment of the disclosure.

FIG. 9A is a bright field image of human induced pluripotent stem cells (hiPSC) under phase contrast microscope transfected and marked with lentivirus particles carrying fusion multi-modal imaging genes in an embodiment of the disclosure.

FIG. 9B is a fluorescent microscope image of hiPSC transfected and marked with lentivirus particles carrying fusion multi-modal imaging genes in an embodiment of the disclosure.

FIG. 10A is a bioluminescence imaging picture.

FIG. 10B is the relationship between fluorescence signal value and the number of cells.

FIG. 11A is a morphological image of hiPSC marked by fusion multi-modal imaging genes in an embodiment of the present disclosure.

FIG. 11B fluorescence imaging of hiPSC reporter gene mRuby2 marked by fusion multi-modal imaging genes in an embodiment of the present disclosure.

FIG. 11C shows that hiPSC reporter gene Gcamp6f marked by fusion multi-modal imaging genes in an embodiment of the present disclosure has no fluorescence under normal conditions.

FIG. 11D shows the fluorescence signal of the hiPSC reporter gene Gcamp6f marked by the fusion multi-modal imaging genes under the treatment of ionomycin in an embodiment of the present disclosure.

FIG. 12A shows a neural network morphology formed by a differentiation of hiPSC marked by the fusion multi-modal imaging genes in an embodiment of the present disclosure.

FIG. 12B fluorescence maintenance of mRuby2 reporter gene in neural network formed by differentiation of hiPSC marked by fusion multi-modal imaging genes in an embodiment of the present disclosure.

FIG. 13A shows that the Gcamp6f reporter gene in the neural network formed by the differentiation of hiPSC marked by the fusion multi-modal imaging genes has no fluorescence under normal conditions in an embodiment of the present disclosure.

FIG. 13B shows that the Gcamp6f reporter gene in the neural network formed by the differentiation of hiPSC marked by the fusion multi-modal imaging genes generates fluorescence under the action of ionomycin (corresponding to the neural network of FIG. 13A) in an embodiment of the present disclosure.

FIG. 13C shows that the Gcamp6f reporter gene in the neural network formed by the differentiation of hiPSC marked by the fusion multi-modal imaging genes has no fluorescence under normal conditions in an embodiment of the present disclosure.

FIG. 13D shows that the Gcamp6f reporter gene in the neural network formed by the differentiation of hiPSC marked by the fusion multi-modal imaging genes generates fluorescence under ATP treatment (corresponding to the neural network of FIG. 13C) in an embodiment of the present disclosure.

FIG. 14 is a quantitative imaging of cell viability (representing the number of living cells) of brain-like organs cultivated by hiPSC marked by fusion multi-modal imaging gene during ZIKA infection in an embodiment of the present disclosure.

FIG. 15A is a phase contrast microscope bright-field image of hiPSC differentiated cardiomyocytes marked with fusion multi-modal imaging genes in an embodiment of the present disclosure.

FIG. 15B is mRuby2 fluorescence imaging of hiPSC differentiated cardiomyocytes marked with fusion multi-modal imaging genes in an embodiment of the present disclosure.

FIG. 15C shows that the reporter gene Gcamp6f in the hiPSC differentiated cardiomyocytes marked by the fusion multi-modal imaging genes has no fluorescence at rest in an embodiment of the present disclosure.

FIG. 15D shows that the reporter gene Gcamp6f in the hiPSC differentiated cardiomyocytes marked by the fusion multi-modal imaging genes generates fluorescence under the beating state of the cells in an embodiment of the present disclosure.

FIG. 16 is a real-time imaging of calcium activity of hiPSC-differentiated cardiomyocytes marked by fusion multi-modal imaging gene during rhythm fluctuation in an embodiment of the present disclosure.

FIG. 17 is a process of method for constructing a fusion imaging gene.

FIG. 18 is a process of a preparation method of lentiviral expression plasmid.

### DETAILED DESCRIPTION OF THE EMBODIMENTS

In order to explain the embodiment of the present disclosure or the technical scheme in the prior art more clearly, in the following description, different “one embodiment” or “an embodiment” do not necessarily refer to the same embodiment. Different embodiments may be replaced or combined, and for those skilled in the art, other embodiments may be obtained according to these embodiments without creative efforts.

One aspect of an embodiment of the present disclosure relate to fusion imaging gene. The fusion imaging gene includes a bioluminescence imaging gene, a fluorescent protein gene and a calcium imaging gene, and the three genes are linked by linkers.

In a specific embodiment, the bioluminescence imaging gene is selected from Nanoluc gene: the fluorescent protein gene is selected from mRuby2 gene; the calcium imaging gene is selected from Gcamp6f gene.

In a specific embodiment, the linkers are selected from a polypeptide chain. The polypeptide chain preferably contains 2-8 amino acids, and more preferably, the amino acids in the polypeptide chain are selected from two, three or four of S (serine), L (leucine), D (aspartic acid) and G (glycine) to constitute a flexible polypeptide chain:

further preferably, the polypeptide chain is selected from S-L-D-S or G-S-S-G.

In a specific embodiment, the Nanoluc gene and the mRuby2 gene are linked by S-L-D-S: mRuby2 gene and Gcamp6f gene are linked by G-S-S-G. According to the research of the embodiment of the disclosure, it is found that the three reporter genes are fused and expressed through S-L-D-S and G-S-S-G flexible polypeptide chains respectively, which ensures the correct folding and activity of each gene product.

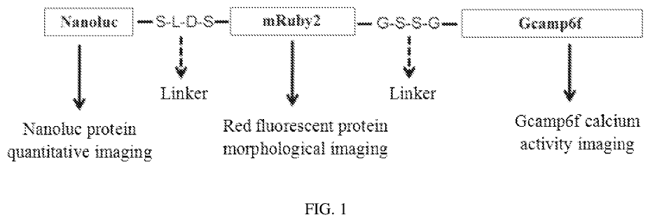

Specifically, the structural schematic diagram of the multi-modal imaging gene is shown in FIG. 1. The fused three reporter genes are Nanoluc for quantitative cellular imaging, mRuby2 for cellular morphological observation and Gcamp6f for cellular calcium activity functional imaging. Nanoluc and mRubi2 protein are linked by S-L-D-S(serine-leucine-aspartic acid-serine), and mRubi2 and Gcamp6f protein are linked by G-S-S-G (glycine-serine-serine-glycine), which may maintain the activity of each protein in the fused protein.

In a specific embodiment, a nucleotide sequence of the fusion imaging gene is shown in SEQ ID NO.1

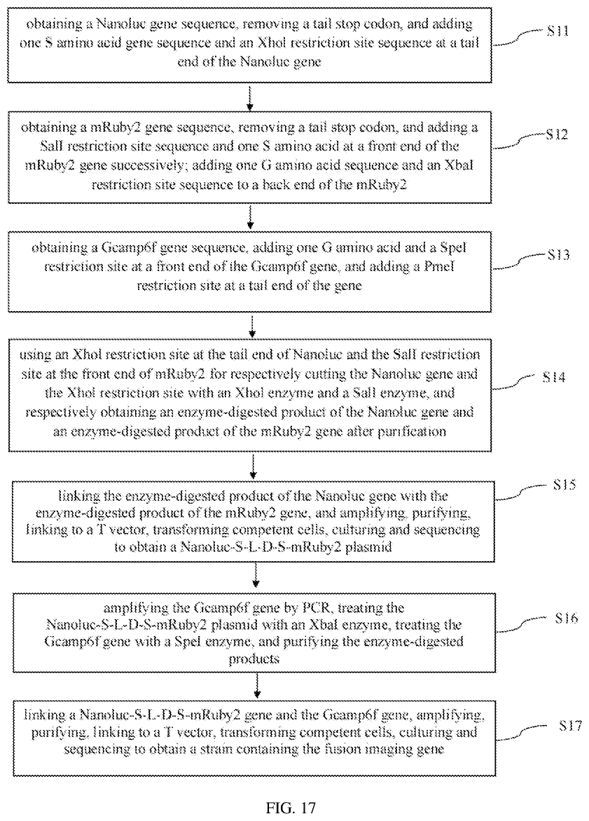

Another aspect of an embodiment of the present disclosure also relates to a method for constructing the fusion imaging gene as shown in FIG. 17, at least including following steps:

S11, obtaining a Nanoluc gene sequence, removing a tail stop codon, and adding one S amino acid gene sequence and an XhoI restriction site sequence at a tail end of the Nanoluc gene, where the Nanoluc gene sequence is synthesized by the gene:

S12, obtaining a mRuby2 gene sequence, removing a tail stop codon, and adding a SalI restriction site sequence and one S amino acid at a front end of the mRuby2 gene successively: adding one G amino acid sequence and an XbaI restriction site sequence to a back end of the mRuby2, where the mRuby2 gene sequence is synthesized by the gene;

S13, obtaining a Gcamp6f gene sequence, adding one G amino acid and a SpeI restriction site at a front end of the Gcamp6f gene, and adding a PmeI restriction site at a tail end of the gene, where preferably the Gcamp6f gene sequence is obtained by Polymerase Chain Reaction (PCR) and more preferably, primer sequences are shown in SEQ ID NO.4 and SEQ ID NO.5:

S14, using the XhoI restriction site at the tail end of Nanoluc and the SalI restriction site at the front end of mRuby2 for respectively cutting the Nanoluc gene and the XhoI gene with an XhoI enzyme and a SalI enzyme, and respectively obtaining an enzyme-digested product of the Nanoluc gene and an enzyme-digested product of the mRuby2 gene after purification:

S15, linking an enzyme-digested product of the Nanoluc gene with an enzyme-digested product of the mRuby2 gene, and amplifying, purifying, linking to a T vector, transforming competent cells, culturing and sequencing to obtain a Nanoluc-S-L-D-S-mRuby2 plasmid:

S16, amplifying the Gcamp6f gene by PCR, treating the Nanoluc-S-L-D-S-mRuby2 plasmid with an XbaI enzyme, treating the Gcamp6f gene with a SpeI enzyme, and purifying the enzyme-digested products: and

S17, linking a Nanoluc-S-L-D-S-mRuby2 gene and the Gcamp6f gene, amplifying, purifying, linking to a T vector, transforming competent cells, culturing and sequencing to obtain a strain containing the fusion imaging gene.

Specific steps are as follows:

(1) Design and Construction of the Fusion Multi-Modal Imaging Gene

1) obtaining Nanoluc gene sequence by gene synthesis, removing the tail stop codon, and adding one S amino acid gene sequence (TCG) and XhoI restriction site sequence at the tail end of Nanoluc gene:

2) obtaining the gene sequence of red fluorescent protein mRuby2 by gene synthesis, removing the tail stop codon, and adding the SalI restriction site sequence (XhoI Isocaudamers) and one S amino acid (TCG) at the front end of mRuby2 successively, adding one G amino acid sequence (GGA) and XbaI restriction site sequence to the back end of mRuby2:

3) amplifying the Gcamp6f gene by PCR, where the plasmid carrying Gcamp6f gene comes from Beijing Maijin Biology Science and Technology Co., Ltd.,; in the primer design, adding one G amino acid (GGA) and a SpeI restriction site (XbaI homotail enzyme) at the front end of the gene successively, and adding a PmeI restriction site at the end of the gene, where the primer sequence is as follows:

SEQ ID NO.4: upstream primer:

actagtggaatgggttctcatcatcatcatcatcatggt (the underlined part is SpeI restriction site and G amino acid codon).

SEQ ID NO.5: downstream primer:

gtttaaactcacttcgctgtcatcatttgta (underlined part is PmeI restriction site);

4) using the XhoI restriction site at the tail end of Nanoluc and the SalI restriction site at the front end of mRuby2 for respectively cutting Nanoluc gene and XhoI gene with XhoI enzyme and SalI enzyme, and purifying the cut products by agarose electrophoresis:

5) utilizing the characteristics that XhoI and SalI are homotail enzymes, connecting the above-mentioned Nanoluc gene enzyme-digested product with the mRuby2 enzyme-digested product:

6) amplifying the ligation product by PCR, recycling and purifying the amplified product by agarose gel electrophoresis, and further linking the purified product to a T vector to transform DH5a competent cells:

7) coating the transformed cells on Luria-Bertani (LB) solid culture medium containing ampicillin and culturing in an incubator at 37° C. for 24 hours:

8) selecting bacterial monoclones, transferring them to test tubes (LB liquid medium containing ampicillin) for amplification and numbering, and culturing until the culture solution is turbid:

9) taking 1 mL of the bacterial liquid in each test tube and submitting it to a sequencing company for sequencing, so as to determine the Nanoluc-S-L-D-S-mRruby2 plasmid with completely correct gene sequence and storing it for later use:

10) amplifying the Gcamp6f gene by PCR, treating the Nanoluc-S-L-D-S-mRuby2 gene with XbaI enzyme, treating the Gcamp6f gene with SpeI enzyme, and purifying the enzyme-digested products by agarose gel electrophoresis; and

11) repeating the above steps of 5)-9), and obtaining the strain containing the fusion gene Nanoluc-S-L-D-S-mRuby2-G-S-S-G-Gcamp6f through ligation, transformation, sequencing, etc., and storing it for later use.

Another aspect of an embodiment of the present disclosure provides a lentiviral expression plasmid of the fusion imaging gene mentioned above. Preferably, a sequence of the lentiviral expression plasmid carrying fusion imaging gene is shown in SEQ ID NO.2.

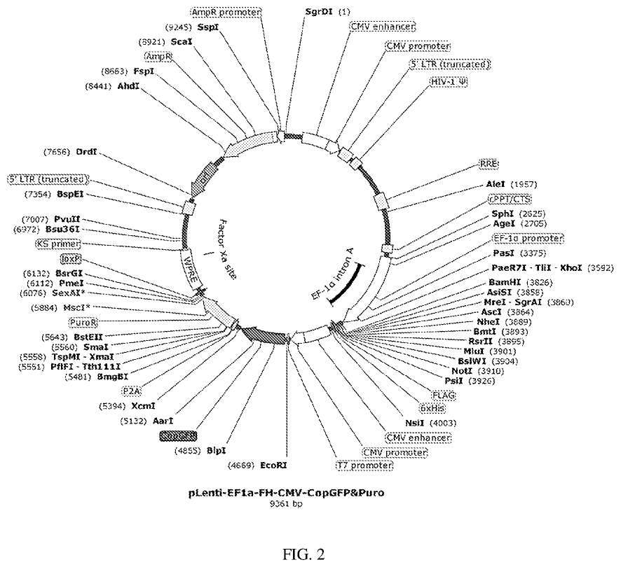

Another aspect of an embodiment of the present disclosure provides a preparation method of lentiviral expression plasmid of fusion imaging gene mentioned above. Because the total length of the fusion multi-modal imaging gene is large, inserting the conventional lentiviral expression plasmid may lead to low success rate of virus packaging and low activity, and it is difficult to carry out cell transfection in the later stage. Therefore, this disclosure takes pLenti-EF1α-FH-CMV-CopGFP&Puro plasmid from Weizhen Bioscience Incorporation as the skeleton (the original plasmid map of lentivirus used for modification is shown in FIG. 2, and the nucleotide sequence is shown in SEQID NO. 3). Firstly, in order to ensure the gene to be widely expressed in different cells, the EF1α promoter is removed by enzyme digestion and replaced by the adult ubiquitination promoter hUbc. Subsequently, the sequence from the downstream of Ubc promoter to Puro gene is deleted by enzyme digestion, which provides more space for the insertion of foreign genes.

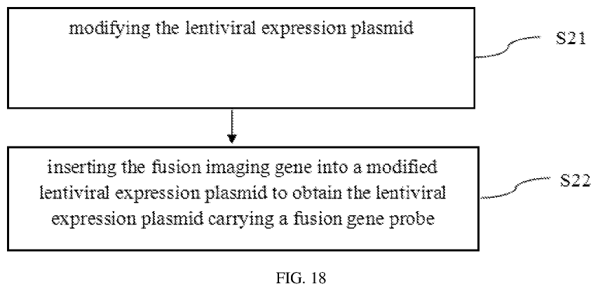

The preparation method of lentiviral expression plasmid at least includes following steps, as shown in FIG. 18:

S21, modifying the lentiviral expression plasmid:

preferably including:

S211, removing an EF-1α promoter in a pLenti-EF1α-FH-CMV-CopGFP&Puro plasmid by double restriction endonuclease digestion with SphI and BamHI to obtain a skeleton plasmid:

S212, inserting a human ubiquitin promoter hUbc into the skeleton plasmid through enzyme link to obtain a pLenti-Ubc-FH-CMV-CopGFP&Puro intermediate plasmid: and S213, removing a sequence from a downstream of Ubc to a Puro gene site in the pLenti-Ubc-FH-CMV-CopGFP&Puro plasmid by PmeI and BamHI double digestion, namely a nucleotide sequence from 3826 to 6112 in the nucleotide sequence shown in the SEQ ID NO.3:

S22, inserting the fusion imaging gene mentioned above into a modified lentiviral expression plasmid to obtain the lentiviral expression plasmid carrying a fusion gene probe:

preferably including:

inserting the fusion imaging gene into the modified intermediate plasmid by using same restriction sites at both ends of the fusion imaging gene to obtain a pLenti-Ubc-Nanoluc-mRuby2-Gcamp6f plasmid, and obtaining the lentiviral expression plasmid through transformation, sequencing, and so on.

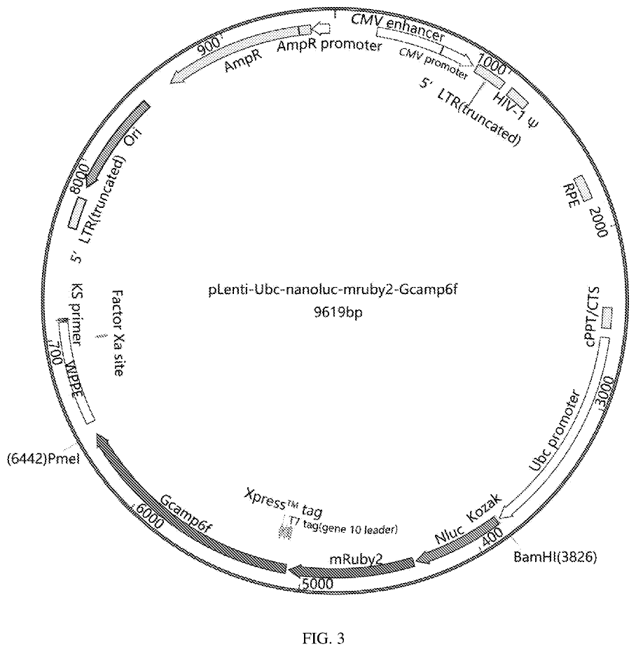

Specifically, the modification of lentiviral expression plasmid and the insertion of fusion imaging gene are mainly realized by the following steps:
1) removing an EF-1α promoter in a pLenti-EF1α-FH-CMV-CopGFP&Puro plasmid by double restriction endonuclease digestion with SphI and BamHI:
2) inserting a human ubiquitin promoter hUbc into the skeleton plasmid through enzyme link to replace the EF-1α promoter, obtaining a pLenti-Ubc-FH-CMV-CopGFP&Puro intermediate plasmid, and obtaining the plasmid with completely correct sequencing through transformation and sequencing:
3) removing a sequence from a downstream of Ubc to a Puro gene site in the pLenti-Ubc-FH-CMV-CopGFP&Puro plasmid by BamHI and PmeI double digestion, namely a nucleotide sequence from 3826 to 6112 in the nucleotide sequence shown in the SEQ ID NO.3:
4) using the prepared NanoLuc-mRuby2-Gcamp6f to fuse the same restriction sites (BamHI and PmeI) at both ends of the multiple reporter genes, and inserting the fusion gene into the restriction plasmid to obtain the pLenti-Ubc-NanoLuc-mRuby2-Gcamp6f plasmid, and obtaining the lentiviral expression plasmid with completely correct sequence through transformation, sequencing, etc. The lentiviral expression plasmid map of the obtained multi-modal imaging gene is shown in FIG. 3. The fusion multi-modal imaging gene is driven by UBC promoter, and the plasmid skeleton is modified to delete the screening genes such as puro and the promoter sequence in the original plasmid in FIG. 2, providing more space for the insertion of foreign genes. The total length of expression plasmid remains below 10000 bp after the insertion of fusion multi-modal imaging gene, which is equivalent to the length of conventional lentiviral expression plasmid.

Another aspect of the embodiment of the disclosure relate to a lentivirus carrying the fusion imaging gene. This lentivirus may be used for transfection marking of different cells to obtain cells stably marked by fusion imaging gene. The cells may be used for multi-modal imaging tracking of different fates of cells, including quantitative imaging, morphological imaging and calcium activity functional imaging.

Another aspect of the embodiment of the present disclosure relates to a preparation method of the lentivirus, and the lentivirus carrying the fusion imaging gene is obtained by co-transfecting cells by the pLenti-Ubc-Nanoluc-mRuby2-Gcamp6f plasmid and lentivirus packaging plasmids and packaging, at least including the following steps:

co-transfecting 293T cells by pLenti-Ubc-Nanoluc-mRuby2-Gcamp6f plasmid and lentivirus packaging plasmids PAX2 and pMD2G to obtain lentivirus particles carrying fusion imaging gene.

Preferably, the mass ratio of pLenti-Ubc-Nanoluc-mRuby2-Gcamp6f plasmid, plasmid PAX2 and plasmid pMD2G is 3: 2: 1.

Another aspect of the embodiment of the present disclosure relates to the cells marked by the fusion imaging gene or the cells infected by the lentivirus. Preferably, the cells are selected from human H1 embryonic stem cells and human pluripotent stem cells.

Another aspect of the embodiment of the disclosure relates to the applications of the cells in simultaneously realizing cell number imaging, morphological imaging and calcium activity function imaging detection.

### Embodiment 1: Preparation of Lentivirus Particles Carrying Fusion Multi-Modal Imaging Gene

(1) Design and Construction of the Fusion Multi-Modal Imaging Gene

1) in order to reduce the influence of fusion expression of multiple imaging genes on protein folding and spatial conformation, and maintain the activity of each protein, Nanoluc, mRuby2 and Gcamp6f are respectively connected by flexible polypeptide sequences S-L-D-S(SEQ ID NO.8) and G-S-S-G (SEQ ID No.9) as linkers, and fusion expression of multiple imaging genes is as shown in FIG. 1.

2) Nanoluc gene and mRuby2 gene are obtained by gene synthesis, respectively, the tail stop codon is removed, BamHI restriction enzyme sequence (GGATCC) is added at the front end of Nanoluc gene, and one S amino acid gene sequence (TCG) and XhoI restriction enzyme sequence (CTCGAG) are added at the end: SalI restriction enzyme sequence (GTCGAC, XhoI homotail enzyme) and one S amino acid (TCG) are successively added to the front end of mRuby2. One G amino acid sequence (GGA) and an XbaI restriction enzyme sequence (TCTAGA) are added to the back end of mRuby2.

3) The plasmid carrying Gcamp6f gene comes from Beijing Maijin Biology Science and Technology Co., Ltd., and the Gcamp6f gene is amplified by PCR. In the primer design, one G amino acid (GGA) and a SpeI restriction site (XbaI homotail enzyme) are successively added at the front end of the gene, and a PmeI restriction site sequence is added at the end of the gene. The primer sequence is as follows:

SEQ ID NO.4: upstream primer:

actagtggaatgggttctctctctctctctctctctctctctgt

(the underline part is SpeI restriction site and G amino acid codon);

SEQ ID NO.5: downstream primer:

gtttaaactcacttcgctgtcatttgta (the underlined part is PmeI restriction site sequence).

4) The XhoI restriction site at the tail end of Nanoluc and the SalI restriction site at the front end of mRuby2 are used to cut the Nanoluc gene and mRuby2 gene by XhoI enzyme and SalI enzyme (purchased from Thermo Company) respectively, and the cut products are purified by agarose electrophoresis: by using the characteristics that XhoI and SalI are homotail enzymes, the above-mentioned enzyme-digested product of Nanoluc gene are connected with the enzyme-digested product of mRuby2: the ligation product is amplified by PCR, amplification product are recycled and purified by agarose gel electrophoresis, the purified product is further ligated to a T vector (purchased from Takara Company) and DH5a competent cells (purchased from Bome Company) are transformed.

5) The transformed cells are coated on LB solid culture medium containing ampicillin and cultured in an incubator at 37° C. for 24 hours: about 5 mL of LB liquid medium containing ampicillin is added into sterile test tubes which are numbered respectively, bacterial monoclones are selected, each clone is transferred to a test tube, and cultured and amplified: 1 mL of bacterial liquid is taken from each of the above test tubes, submitted to a sequencing company for sequencing to determine the bacterial liquid of Nanoluc-S-L-D-S-mRuby2 with completely correct gene sequence, amplify and extract the plasmid, and store for later use.

6) PCR amplification of Nanoluc-S-L-D-S-mRuby2 gene,

SEQ ID NO.6: upstream primer:

ggatccatggtcttcacactcgaagatttcgt

(the underline part is BamHI restriction sequence),

SEQ ID NO.7: downstream primer:

tctagatcccttgtacagctcgtccatcccaccac

(the underlined part is XbaI restriction site);

XbaI enzyme (Thermo Company) is used to treat PCR products, while SpeI enzyme (purchased from Thermo Company) is used to treat Gcamp6f gene amplification products, and the enzyme-digested products are purified by agarose gel electrophoresis.

7) Accord to the same method as in the previous step 4)-5), a bacterial solution containing the fusion gene Nanoluc-S-L-D-S-mRuby2-G-S-S-G-Gcamp6f with completely correct sequence is obtained through ligation, transformation, sequencing and the like, and the plasmid (T vector plasmid) is amplified and extracted, and stored for later use. As shown in SEQ ID NO.1, the sequence of fusion imaging gene is constructed.

(2) Construction of Lentiviral Expression Plasmid Carrying Fusion Imaging Gene

1) taking the pLenti-EF1α-FH-CMV-CopGFP&Puro plasmid from Weizhen Bioscience Incorporation as the skeleton (as shown in FIG. 2, the nucleotide sequence is shown in SEQ ID NO.3), and removing the EF-1α promoter in the original plasmid by double restriction endonuclease digestion with SphI and BamHI (purchased from Thermo Company);

2) inserting the human ubiquitin promoter hUbc into the skeleton plasmid through enzyme connection to replace the EF-1α promoter, and obtaining the pLenti-Ubc-FH-CMV-CopGFP &Puro plasmid:

3) transforming DH5a competent cells with enzyme ligation products: coating the transformed cells on LB solid medium containing ampicillin and culturing in an incubator at 37° C. for 24 hours: add about 5 mL of LB liquid medium containing ampicillin into sterile test tubes, numbering them respectively, selecting bacterial monoclones, transferring each clone to a test tube, and culturing and amplifying: taking 1 mL of the bacterial solution of each test tube, submitting it to a sequencing company for sequencing to determine the bacterial solution with completely correct gene sequence, amplifying, extracting plasmid, and storing it for later use;

4) using BamHI and PmeI double enzyme digestion to remove the sequence from Ubc downstream to Puro gene site in pLenti-Ubc-FH-CMV-CopGFP&Puro plasmid, that is, the nucleotide sequence from 3826 to 6112 in the nucleotide sequence shown in SEQ ID NO.3:

5) using the same restriction sites (BamHI and PmeI) at both ends of the NanoLuc-mRuby2-Gcamp6 fusion imaging gene prepared above to digest the T vector plasmid containing the above-mentioned fusion gene, so as to obtain the fusion gene fragment, and then connecting the fusion gene by DNA ligase (purchased from Promega Company), and inserting the fusion gene into the downstream of Ubc promoter in the above-mentioned restriction plasmid to obtain pLenti-Ubc-Nanoluc-mRuby2-Gcamp6f plasmid:

6) as in the above step 3) to obtain the plasmid with completely correct gene sequence and store for later use.

As shown in FIG. 3, the structural map of lentiviral expression plasmid carrying fusion gene is successfully constructed, and the sequence of plasmid gene is shown in SEQ ID NO.1

(3) Preparation of Lentivirus Carrying the Fusion Multi-Modal Imaging Gene

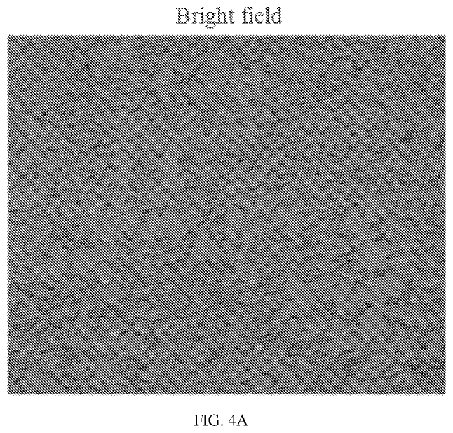

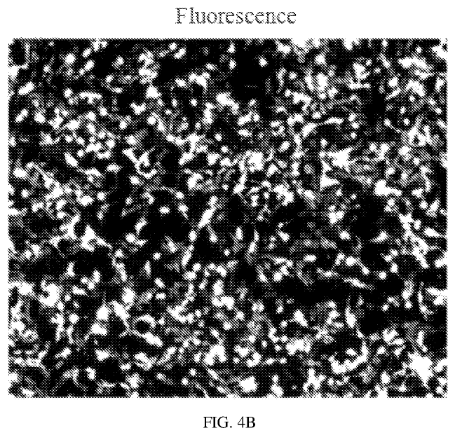

The above-mentioned pLenti-Ubc-Nanoluc-mRuby2-Gcamp6f plasmid, packaging plasmids PAX2 and PMD2G in the ratio of 3:2:1 are used to co-transfect 293T cells according to the conventional lentivirus packaging method (refer to PLOS One 2013: 8(6): e66369). After 48 hours of transfection, the transfection efficiency of fluorescent protein is observed to preliminarily judge the packaging efficiency. The experimental results are shown in FIG. 4A-FIG. 4B. FIG. 4A-FIG. 4B show the efficiency of co-transfecting 293T cells by the modified lentiviral expression plasmid into which the multi-modal imaging gene inserted (the total length of the plasmid is less than 10000 bp) together with the packaging plasmids. Co-transfection of 293T cells by the lentiviral expression plasmid with the multi-modal imaging gene and the packaging plasmids has relatively high transfection efficiency, and from the observation of fluorescence expression rate, the successful transfection rate is 80-90%, suggesting that it has good virus packaging efficiency.

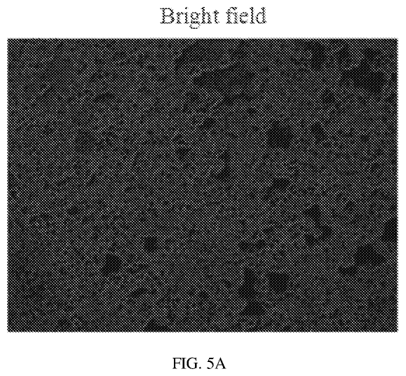

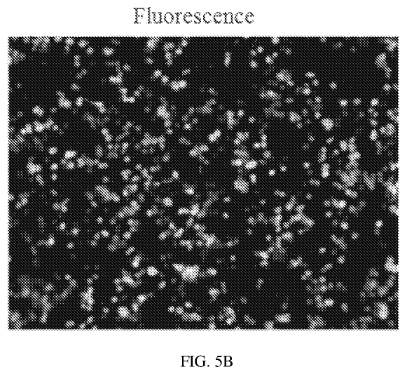

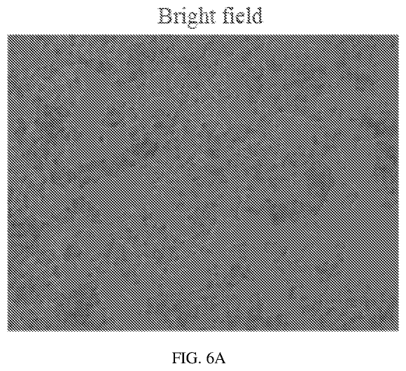

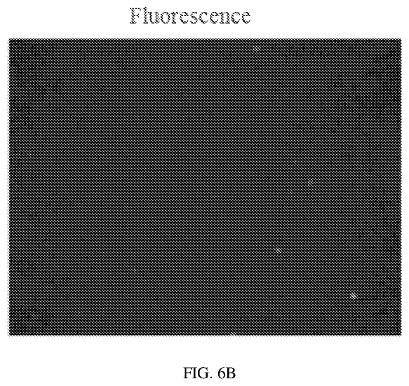

At the same time, the conventional lentiviral expression plasmid carrying a single fluorescent protein (only carrying mRuby2 gene) and the conventional lentiviral expression plasmid carrying a plurality of imaging genes with a long length (using pLenti-EF1α-FH-CMV-CopGFP&Puro plasmid before modification to insert the fusion multi-modal imaging gene in embodiment 1 of the present disclosure directly) are used as controls to compare the virus packaging efficiency. FIG. 5A-FIG. 5B show the efficiency of co-transfection of 293T cells by the conventional lentiviral expression plasmid into which the single reporter gene is inserted (the total length of the plasmid is less than 10000 bp) together with the packaging plasmids. It can be seen from FIG. 5A-FIG. 5B that the efficiency of co-transfection of 293T cells is about 80-90% when the conventional lentivirus is packaged, which is equivalent to the efficiency of the modified plasmid packaging virus inserted with multi-modal imaging gene. FIG. 6A-FIG. 6B show the efficiency of co-transfection of 293T cells by the general lentiviral expression plasmid into which the fusion multi-modal imaging gene is inserted (the total length of the plasmid is >11000 bp), together with the packaging plasmids.

As shown in FIG. 4A-FIG. 4B, the co-transfection efficiency of the modified plasmid carrying the fusion gene in the embodiment of the present disclosure is 80-90%, which is equivalent to that of the conventional plasmid carrying a single fluorescent protein as shown in FIG. 5A-FIG. 5B, suggesting that both the lentiviral plasmid inserted with multiple reporter genes and the conventional lentiviral plasmid have higher lentiviral packaging efficiency, while as shown in FIG. 6A-FIG. 6B, the co-transfection packaging efficiency of the conventional lentiviral plasmid inserted with multiple reporter genes and with long length is very low, about 10-20%. The lentiviral expression plasmid with long length deadly influences the virus packing rate. The results show that it is necessary to modify the conventional lentivirus plasmid skeleton, provide space for the longer fusion gene probe and reduce the total length of plasmid, so as to maintain the packaging efficiency of lentivirus. The deletion and modification of plasmid skeleton in the disclosure improves the length of foreign gene insertion and maintains plasmid activity.

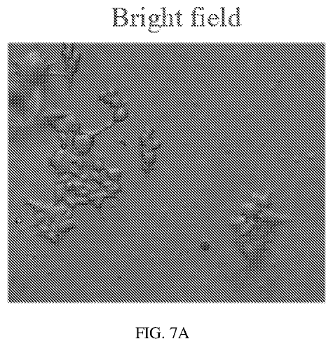

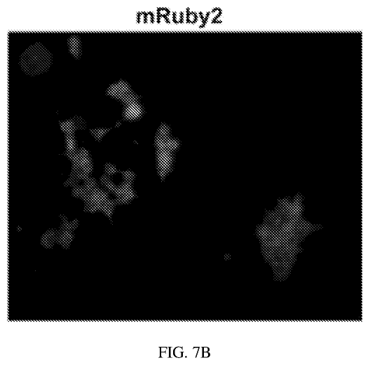

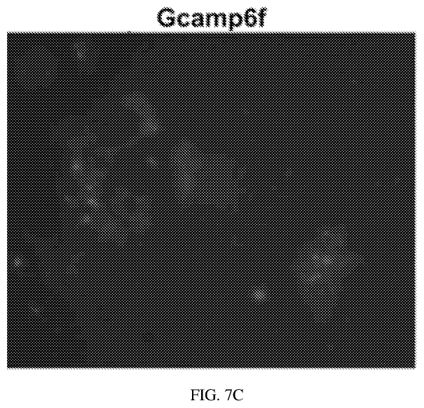

After lentivirus particles are collected, 293T cells are marked with transgene by conventional lentivirus infection. The experimental results are shown in FIG. 7A-FIG. 7C. As can be seen from FIG. 7A-FIG. 7C, 293T cells are successfully infected by the virus and expressed by mRuby2 fluorescent protein, while Gcamp6f is basically in a non-fluorescent state in a conventional environment without calcium ions. It is proves that lentivirus has good activity, and the normal activity of each protein is maintained by connecting the fusion gene with the peptide chain of S-L-D-S and G-S-S-G, suggesting that lentivirus particles carrying multi-modal imaging gene prepared by the disclosure have good activity.

### Embodiment 2: Application of Fusion Multi-Modal Imaging Gene in Human Pluripotent Stem Cells

(1) Multi-Modal Imaging Gene Marker of Human Embryonic Stem Cells (hESC)

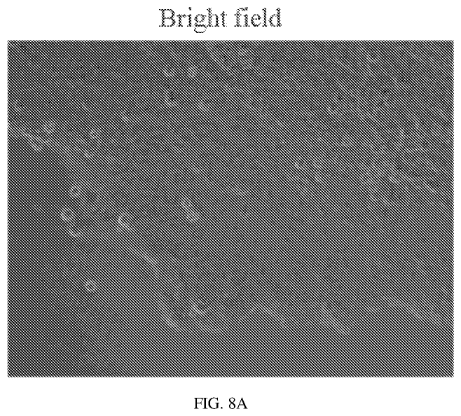

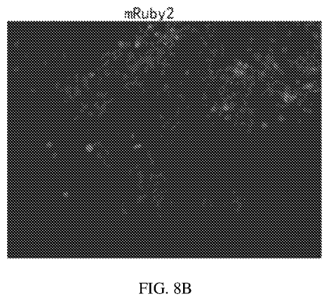

h1ESC (presented by Professor Cao Nan of Sun Yat-sen University or purchased from American ATCC) is transfected by the lentivirus particles carrying the fusion gene probe mentioned above through infection method of cells by conventional lentiviruses (refer to PLOS One. 2013: 8(6):e66369), and the positive clones are isolated and amplified by the method described by the patent (ZL201911133148.1). The experimental results are shown in FIG. 8A-FIG. 8B. From FIG. 8A-FIG. 8B, it can be seen that the lentivirus particles carrying the fusion multi-modal imaging gene prepared by the disclosure successfully transfect hESC, and the expression of mRuby2 is observed, thus successfully obtaining the hESC cell line marked by the fusion imaging gene. It is suggested that the lentivirus particles carrying multi-modal imaging gene prepared by the disclosure have good activity.

(2) Multi-Modal Imaging Gene Marker of Human Induced Pluripotent Stem Cells (hiPSC)

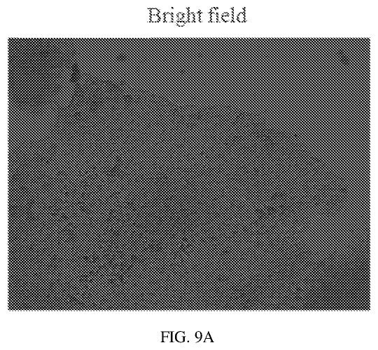

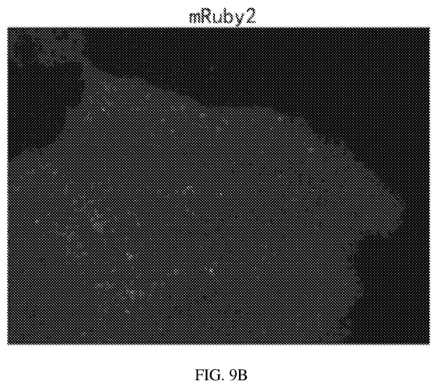

The lentivirus particles carrying the fusion gene probe is used to transfect hiPSC0100 (from Shanghai Stem Cell Bank of Chinese Academy of Sciences) through infection method of cells by conventional lentiviruses (refer to PLOS One. 2013: 8(6): e66369), and the positive clones are isolated for amplification by the method described by patented (ZL201911133148.1). The experimental results are shown in FIG. 9A-FIG. 9B. From FIG. 9A-FIG. 9B, it can be seen that the lentivirus particles carrying the fusion multi-modal imaging gene prepared by the disclosure successfully transfects hiPSC, and the expression of mRuby2 is observed, and the hiPSC cell line marked by the fusion imaging gene is successfully obtained. It is suggested that the lentivirus particles carrying multi-modal imaging gene prepared by the disclosure have good activity.

(3) Multi-Modal Imaging Detection of Human Pluripotent Stem Cells

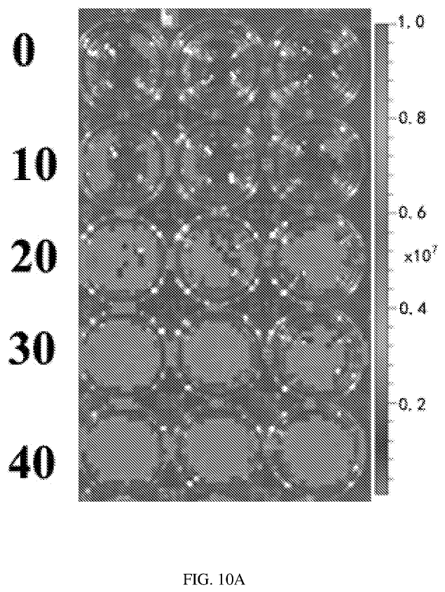

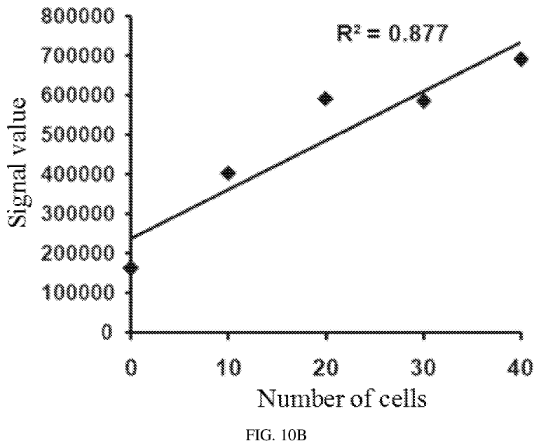

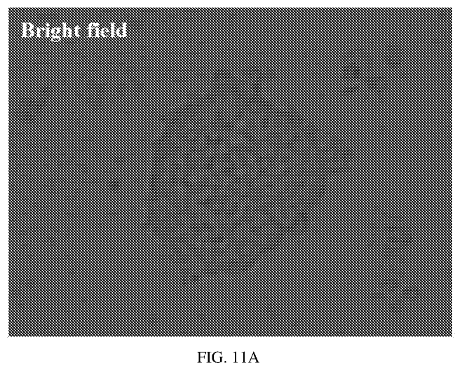

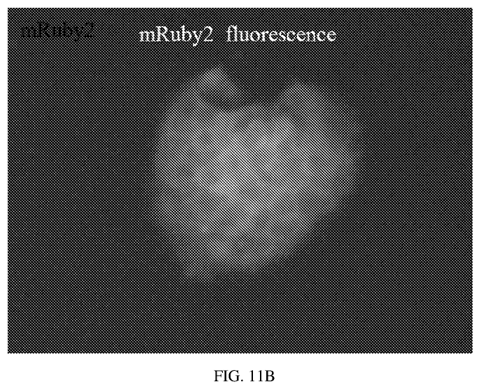

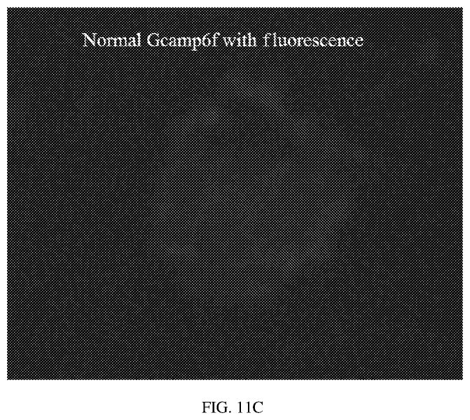

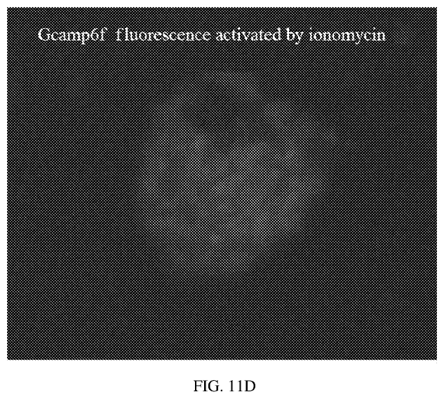

Quantitative imaging, morphological imaging and calcium activity functional imaging are carried out by using hiPSC marked by fusion imaging gene. The basic method of quantitative imaging is to collect marked hiPSC with good growth state, disperse it into single cell suspension, dilute it to 0, 10, 20, 30, 40 iPSC per 100 uL of culture solution, 100 uL of the above gradient cell suspension is add to a 96-well culture plate, Nanoluc substrate solution is added according to the instructions of the kit (from Promega, article number N1110), and an in vivo imager (Invitrogen in vivo) is used for detecting fluorescence signal. The experimental results are shown in FIG. 10A-FIG. 10B, in which FIG. 10A is a bioluminescence imaging picture, and FIG. 10B is the relationship between fluorescence signal value and the number of cells. It shows that bioluminescence imaging based on Nanoluc may quantify the number of cells, and the sensitivity may reach less than 10 cells. The results of morphological imaging (mRuby2) and calcium activity functional imaging (Gcamp6f) are shown in FIG. 11A-FIG. 11D. The typical morphology of cells are observed by fluorescent microscope imaging by using mRuby2, and the morphology of marked cells are detected after cell transplantation: at the same time, it is observed that Gcamp6f is in a state of no or low fluorescence under normal conditions. By adding 10 UM ionomycin (purchased from Sigma Company) to stimulate calcium activity, the fluorescence of Gcamp6f is significantly activated, indicating that the fusion imaging gene is capable of cellular calcium activity imaging.

### Embodiment 3: Application of Fusion Multi-Modal Imaging Gene in Human Pluripotent Stem Cells-Differentiated Cells

(1) Morphological Imaging Detection of Nerve Cells Derived from hiPSC

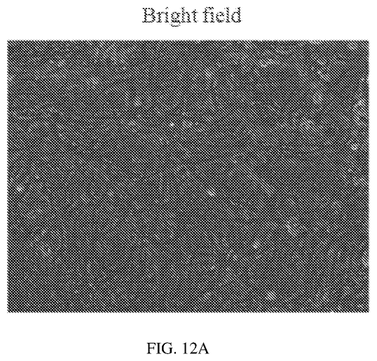

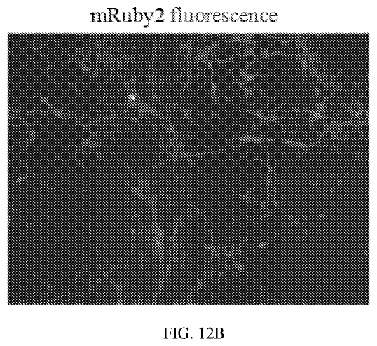

According to the existing methods (English book Methods in Molecular Biology, Neural Stem Cells PP 1-7), the hiPSC is differentiated into neural stem cells (NSCs), and then NSC balls are attached to the wall for growth, and the neuron culture medium is used for long-term culture to promote the differentiation into nerve cells. The experimental observation results of morphological imaging of neural network formed by hiPSC differentiation are shown in FIG. 12A-FIG. 12B. It can be seen from FIG. 12A-FIG. 12B that NSC differentiated and cultured for 4 months forms an obvious neural network morphology, and the stable expression of mRuby2 is observed through a fluorescent microscope (purchased from Nikon Company, Ti2-U model), showing a good neural network morphology. It is suggested that multi-modal imaging gene may still maintain stable expression after cell proliferation and nerve differentiation, and may be used for stable imaging tracking of differentiated nerve cells.

(2) Functional Imaging Detection of Calcium Activity in Nerve Cells Derived from hiPSC

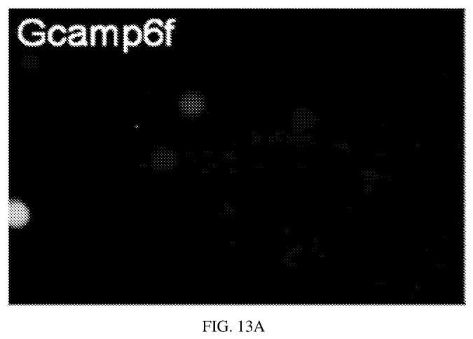

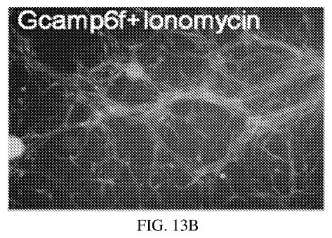

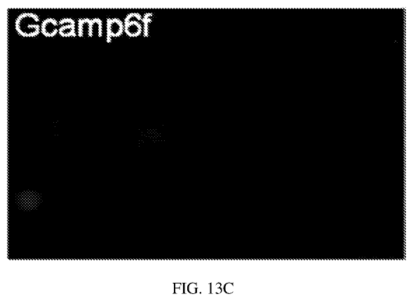

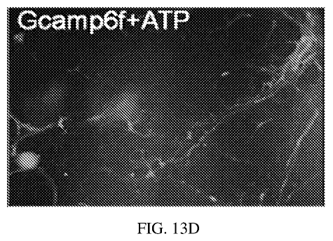

Using the neural network formed by the above-mentioned hiPSC differentiation, the reaction of Gcamp6f protein is observed under the normal environment and the stimulation of ionomycin and ATP respectively. The experimental results of calcium activity functional imaging of neural network formed by hiPSC differentiation are shown in FIG. 13A-FIG. 13D. As shown in FIG. 13A-FIG. 13D, the activation of fluorescence signal is observed under the stimulation of ionomycin and ATP (purchased from Sigma Company), which indicates that the fusion gene probe maintains good activity in hiPSC differentiation and may be used for imaging calcium activity of differentiated cells.

(3) Real-Time Quantitative Imaging Detection of Cell Viability in Brain-Like Organs Derived from hiPSC

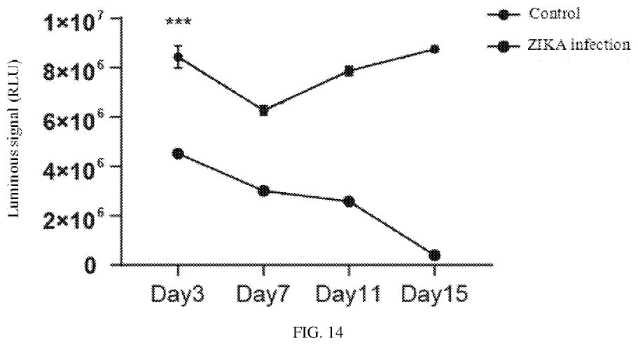

According to the method reported in the literature (J. Vis. Exp. 2017; 127: e56404), iPSC marked by fusion gene probe is cultivated to form brain-like organs as the infection model of ZIKA. Virus infection may lead to nerve cells and neural stem cells to die, but it can't be distinguished by conventional morphological observation. Previous studies have used invasive sampling to detect. Using Nanoluc protein, the cell viability is detected non-invasively and continuously at different time points after ZIKA infection, and the experimental results of cell number imaging of brain-like organs cultivated by hiPSC during ZIKA infection are shown in FIG. 14. As shown in FIG. 14, three days after ZIKA infection, the cells in brain-like organs die obviously, and then the cell vitality continues to decline during the infection process, thus realizing continuous, visual and quantitative imaging of the cell vitality in the same specimen.

(4) Real-time imaging detection of calcium activity in myocardial cells derived from hiPSC

1) fusing gene-marked hiPSC cells, culturing until 80% confluence:

2) replacing by RPMI1640 medium (purchased from Gibco company), adding insulin-free B27 (1X, purchased from Gibco company) and 6 uM CHIR99021 (purchased from Merck company), and culturing for 48 hours;

3) replacing by fresh RPMI1640 medium, adding insulin-free B27 (1X), and culturing for 24 hours:

4) replacing by RPMI1640 medium, adding insulin-free B27 (1X) and 6 uM IWR-1 (purchased from Merck company), and culturing for 48 hours:

5) replacing by RPMI1640 medium, adding insulin-free B27(1X) and culturing for 48 hours:

6) replacing RPMI1640 medium, adding B27 (1X) containing insulin, and changing the liquid every three days. The appearance of pulsatile cells is observed during culture:

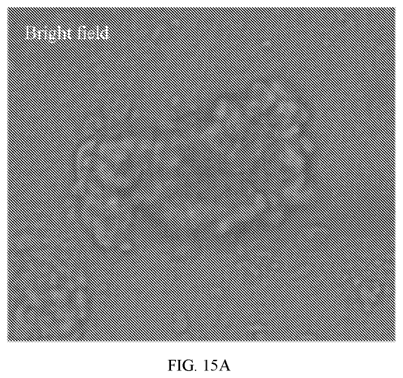

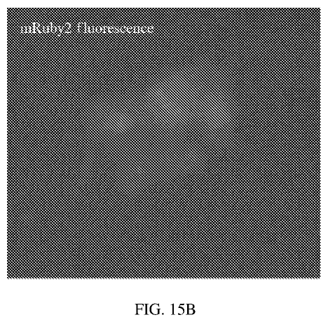

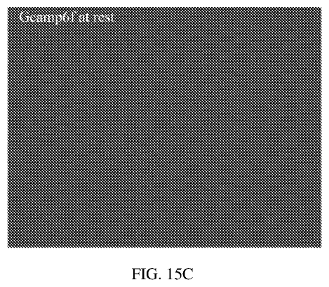

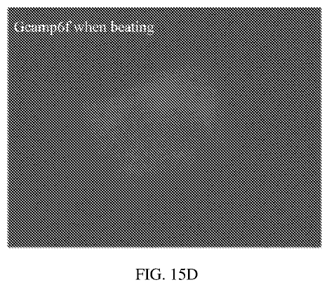

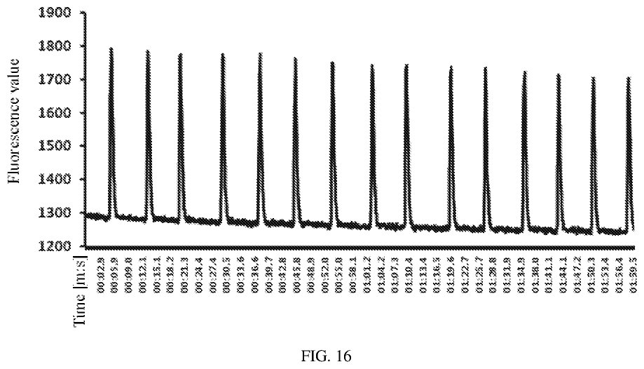

7) using living cell workstation (purchased from Nikon Company) to observe the expression of mRuby2: at the same time, recording the fluorescence changes of Gcamp6f in real time. The experimental results of morphological imaging and calcium activity functional imaging of hiPSC differentiated cardiomyocytes are shown in FIG. 15A-FIG. 15D. The stable expression of mRuby2 is observed by using marked iPSC differentiated cardiomyocytes, and at the same time, it can be observed from Gcamp6f imaging that cardiomyocytes have no fluorescence at rest and activate fluorescence sensitively when beating. Furthermore, in the process of myocardial beating, the rhythm change of fluorescence signal may be detected when myocardial cells beat rhythmically. The experimental results of continuous imaging of calcium activity of hiPSC differentiated nerve cells marked by multi-modal imaging gene are shown in FIG. 16. As shown in FIG. 16, the marked iPSC is used to differentiate and form cardiomyocytes, which are accompanied by the rhythm change of calcium activity in the beating process, and the Gcamp6f and the living cell workstation are used for real-time imaging, so that the rhythm change of fluorescence signals accompanied by the rhythm fluctuation of cardiomyocytes may be observed, which indicates that the multi-modal imaging gene in the disclosure may be used for high-sensitivity and real-time imaging detection of cellular calcium activity.

What is claimed is:

1. A fusion imaging gene, wherein the fusion imaging gene comprises a bioluminescence imaging gene, a fluorescent protein gene and a calcium imaging gene, and the three genes are linked by linkers;
the bioluminescence imaging gene is selected from a Nanoluc gene;
the fluorescent protein gene is selected from a mRuby2 gene;
the calcium imaging gene is selected from a Gcamp6f gene;
the Nanoluc gene and the mRuby2 gene are linked through S-L-D-S: the mRuby2 gene and the Gcamp6f gene are linked through G-S-S-G;
a nucleotide sequence of the fusion imaging gene is shown in SEQ ID NO.1.

2. A method for constructing the fusion imaging gene according to claim 1, at least comprising following steps:
S11, obtaining a Nanoluc gene sequence, removing a tail stop codon, and adding one S amino acid gene sequence and an XhoI restriction site sequence at a tail end of the Nanoluc gene;
S12, obtaining a mRuby2 gene sequence, removing a tail stop codon, and adding a SalI restriction site sequence and one S amino acid at a front end of the mRuby2 gene successively: adding one G amino acid sequence and an XbaI restriction site sequence to a back end of the mRuby2;
S13, obtaining a Gcamp6f gene sequence, adding one G amino acid and a SpeI restriction site at a front end of the Gcamp6f gene, and adding a PmeI restriction site at a tail end of the gene;
S14, using an XhoI restriction site at the tail end of Nanoluc and the SalI restriction site at the front end of mRuby2 for respectively cutting the Nanoluc gene and the XhoI restriction site with an XhoI enzyme and a SalI enzyme, and respectively obtaining an enzyme-digested product of the Nanoluc gene and an enzyme-digested product of the mRuby2 gene after purification;
S15, linking the enzyme-digested product of the Nanoluc gene with the enzyme-digested product of the mRuby2 gene, and amplifying, purifying, linking to a T vector, transforming competent cells, culturing and sequencing to obtain a Nanoluc-S-L-D-S-mRuby2 plasmid;
S16, amplifying the Gcamp6f gene by PCR, treating the Nanoluc-S-L-D-S-mRuby2 plasmid with an XbaI enzyme, treating the Gcamp6f gene with a SpeI enzyme, and purifying the enzyme-digested products; and
S17, linking a Nanoluc-S-L-D-S-mRuby2 gene and the Gcamp6f gene, amplifying, purifying, linking to a T vector, transforming competent cells, culturing and sequencing to obtain a strain containing the fusion imaging gene.

3. The method for constructing the fusion imaging gene according to claim 2, wherein in the S11, the Nanoluc gene sequence is synthesized by the gene.

4. The method for constructing the fusion imaging gene according to claim 2, wherein in the S12, the mRuby2 gene sequence is synthesized by the gene.

5. The method for constructing the fusion imaging gene according to claim 2, wherein in the S13, the Gcamp6f gene sequence is obtained by PCR.

6. The method for constructing the fusion imaging gene according to claim 5, wherein in the S13, primer sequences are shown in SEQ ID NO.4 and SEQ ID NO.5.

7. A lentiviral expression plasmid carrying the fusion imaging gene according to claim 1.

8. The lentiviral expression plasmid of fusion imaging gene according to claim 7, wherein a sequence of the lentiviral expression plasmid carrying fusion imaging gene is shown in SEQ ID NO.2.

9. A preparation method of lentiviral expression plasmid, at least comprising following steps:
S21, modifying the lentiviral expression plasmid; and
S22, inserting the fusion imaging gene according to claim 1 into a modified lentiviral expression plasmid to obtain the lentiviral expression plasmid carrying a fusion gene probe.

10. The preparation method of lentiviral expression plasmid according to claim 9, wherein in the S21, the modifying comprises:
S211, removing an EF-1α promoter in a pLenti-EF1α-FH-CMV-CopGFP&Puro plasmid by double restriction endonuclease digestion with SphI and BamHI to obtain a skeleton plasmid;
wherein a nucleotide sequence of the pLenti-EF1α-FH-CMV-CopGFP&Puro plasmid is shown in SEQ ID NO.3;
S212, inserting a human ubiquitin promoter hUbc into the skeleton plasmid through enzyme link to obtain a pLenti-Ubc-FH-CMV-CopGFP&Puro intermediate plasmid; and
S213, removing a sequence from a downstream of Ubc to a Puro gene site in the pLenti-Ubc-FH-CMV-CopGFP&Puro plasmid by PmeI and BamHI double digestion, namely a nucleotide sequence from 3826 to 6112 in the nucleotide sequence shown in the SEQ ID NO.3.

11. The preparation method of lentiviral expression plasmid according to claim 9, wherein in the S22, the fusion imaging gene are inserted into the modified intermediate plasmid by using same restriction sites at both ends of the fusion imaging gene to obtain a pLenti-Ubc-Nanoluc-mRuby2-Gcamp6f plasmid, and the lentiviral expression plasmid is obtained through transformation, sequencing, and so on, and a nucleotide sequence of the lentiviral expression plasmid is shown in SEQ ID NO.2.

12. A lentivirus carrying the fusion imaging gene according to claim 1.

13. A preparation method of the lentivirus according to claim 12, at least comprising following steps:
co-transfecting 293T cells by the plasmid shown in the SEQ ID NO.2 and lentivirus packaging plasmids PAX2 and pMD2G to obtain lentivirus particles carrying the fusion imaging gene.

14. The preparation method of the lentivirus according to claim 13, wherein a mass ratio of the plasmid shown in the SEQ ID NO.2, the plasmid PAX2 and the plasmid pMD2G3 is 3-4: 1.5-2.5: 0.5-1.5.

15. The preparation method of the lentivirus according to claim 13, wherein the mass ratio of the plasmid shown in the SEQ ID NO.2, the plasmid PAX2 and the plasmid pMD2G3 is 3: 2: 1.

16. A cell marked by the fusion imaging gene according to claim 1.

17. A cell marked by the fusion imaging gene according to claim 1, wherein the cell is a human H1 embryonic stem cell or a human pluripotent stem cell.
